(19) United States
(12) Patent Application Publication
(10) Pub. No.: US 2021/0327541 A1
(43) Pub. Date: Oct. 21, 2021

(54) DETECTION METHOD AND DETECTION APPARATUS FOR GENOMIC STRUCTURAL VARIATIONS BASED ON K-MER SET IN REFERENCE GENOME

(71) Applicant: INDUSTRY-UNIVERSITY COOPERATION FOUNDATION HANYANG UNIVERSITY, Seoul (KR)

(72) Inventors: Jin Wu NAM, Seoul (KR); Minhak CHOI, Seoul (KR); Dohun YI, Seoul (KR); Jang-il SOHN, Gwangju-si (KR)

(73) Assignee: INDUSTRY-UNIVERSITY COOPERATION FOUNDATION HANYANG UNIVERSITY, Seoul (KR)

(21) Appl. No.: 17/272,383

(22) PCT Filed: Nov. 16, 2018
(86) PCT No.: PCT/KR2018/014079

§ 371 Date: Mar. 1, 2021

(30) Foreign Application Priority Data
Sep. 28, 2018 (KR) 10-2018-0116410
Nov. 14, 2018 (KR) 10-2018-0139875

(51) Int. Cl. G16B 40/00; G16B 20/20; G16H 50/50; G06N 5/02

(52) U.S. Cl. CPC G16B 40/00; G06N 5/02; G16H 50/50; G16B 20/20

(57) ABSTRACT

Disclosed is a method of detecting a genomic structural variation based on k-mer set in a reference genome by means of a computer apparatus, the method including receiving sample sequence data, comparing the sample sequence data to k-mer set in reference genome data to determine at least one k-mer read that is not included in the reference genome data among reads of the sample sequence data, determining a breakpoint and a candidate region of a structural variation by mapping the at least one k-mer read to standard reference genome data, and predicting a structural variation type for the sample sequence data on the basis of a sequence mapping pattern and the breakpoint corresponding to the mapping result.

### TECHNICAL FIELD

The following description relates to a technique for detecting genomic structural variations.

### BACKGROUND ART

Genomic variations may be largely divided into sequence variations and structural variations. Structural variations refer to genetic segmental duplication greater than or equal to 1000 base pairs (bp; length of nucleic acid), copy number variation, translocation, inversion, insertion, or deletion.

Recently, along with the development of next-generation sequencing (NGS), techniques for discovering structural variations using sequence fragments (reads) generated by a sequencing apparatus have been introduced. For sequence variation analysis, various efficient algorithms have emerged based on large-scale sequence data. On the other hand, structural variation prediction, which has much higher complexity, has no market-dominant algorithm or program in terms of performance and speed.

### DISCLOSURE

### Technical Problem

Prediction of structural variations in cancer and major diseases is clinically urgent. In particular, as medical insurance is applied to the use of cancer genome panels in Korea, next-generation sequence data is being produced from a large number of cancer patients. However, a technique for predicting or classifying cancer-related structural variations is not supported.

Conventional commercial genomic structural variation analysis programs have limitations in detecting various types of structural variations. For example, BreakDancer is limited in detecting an insertion type because a structural variation is predicted using only information on discordant paired-end reads. Furthermore, the conventional analysis programs have a problem (false positive or false negative) in which a sequence difference due to racial differences is misinterpreted as a sequence associated with a structural variation because genome sequence differences (SNP) between individuals are not considered.

The following description is intended to provide a technique of detecting all types of structural variations through NGS-based analysis. Also, the following description is intended to provide a technique of detecting genomic structural variations in consideration of a genomic sequence difference due to racial differences.

### Technical Solution

A method of detecting a genomic structural variation based on a multi-reference genome includes receiving sample sequence data by a computer apparatus, comparing, by the computer apparatus, the sample sequence data to multi-reference genome data to determine at least one k-mer read that is not included in the multi-reference genome among reads of the sample sequence data, determining, by the computer apparatus, a breakpoint and a candidate region of a structural variation by mapping the at least one k-mer read to standard reference genome data, and predicting, by the computer apparatus, a structural variation type for the sample sequence data on the basis of a sequence mapping pattern and the breakpoint corresponding to the mapping result.

An apparatus for detecting a genomic structural variation based on a multi-reference genome includes an input device configured to receive sample sequence data, a storage device configured to store multi-reference genome data, standard reference genome data, and a program for comparing the multi-reference genome data and the standard reference genome data to the sample sequence data and predicting a structural variation type for the sample sequence data, and a computing device configured to compare the multi-reference genome data and the sample sequence data to determine at least one k-mer read that is not included in the multi-reference genome among reads of the sample sequence data and configured to predict the structural variation type on the basis of a sequence mapping pattern and a breakpoint determined by mapping the at least one k-mer read to the standard reference genome data.

### Advantageous Effects

With the technique described below, it is possible to effectively detect various structural variations using a complex mapping technique. Also, with the technique described below, it is possible to solve an erroneous detection problem due to sequence differences between races by using a complex reference genome in the detection of the genomic structural variation. The technique described below is a genome analysis technique usable for NGS-based cancer diagnosis panels, whole-genome sequencing (WGS), whole-exome sequencing (WES), and targeted panel sequencing (TPS). Furthermore, with the technique described below, it is possible to detect NGS-based germ cell-related structural variations (hereditary) and somatic cell-related structural variations (non-hereditary).

### DESCRIPTION OF DRAWINGS

FIG. 1 is a result of comparing a 31-mer of the hg19 reference genome to 31-mers of reference genomes of various races.

FIG. 2 is an exemplary flowchart of a process of detecting genomic structural variations based on a multi-reference genome.

FIG. 3 is an example of a k-mer filtering result for samples of the 1000 Genomes Project.

FIG. 4 is an example of a k-mer filtering result for a breast cancer sample verified to have structural variations.

FIG. 5 is an example of experimental results for verifying the effect of detection of structural variations.

FIG. 6 is an example of experimental results for verifying the effect of detection of structural variations according to sequencing depth.

FIG. 7 is an example of experimental results for verifying the effect of detection of structural variations according to tumor purity.

FIG. 8 is an example of a structure of a structural variation detection apparatus.

FIG. 9 is an example of a structural variation detection system.

### MODE FOR CARRYING OUT THE INVENTION

As the following description may be variously modified and have several example embodiments, specific embodiments will be shown in the accompanying drawings and described in detail below. It should be understood, however, that there is no intent to limit the following description to the particular forms disclosed, but on the contrary, the following description is to cover all modifications, equivalents, and alternatives falling within the spirit and scope of the invention.

Analysis techniques and terms used herein will be described below.

The NGS-based analysis includes a single-end library method and a paired-end library method. In general, the paired-end technique is more useful for discovering genomic structural variations because two sequence fragments of a sample genome specimen are mapped and compared to a reference genome sample.

A paired-end mapping (PEM)-based structural variation detection technique uses paired-end reads. Two paired reads generated in a genome (case) to be detected have information on a distance from each other. For reference, generally, for genome analysis, a patient group is marked “case,” and a normal group is marked “control.” When two reads are mapped to a reference genome whose sequence is already known, a structural variation is detected by computing the difference between an actual mapping distance to the reference genome and a distance to the case. In this case, since the reads are mapped to the reference genome in consideration of both forward and reverse directions, inversion detection is possible. PEM-based techniques that find and analyze paired reads supports much higher resolution than single-end mapping-based methods. The PEM-based structural variation detection technique analyzes a form in which two reads are mapped. The form or feature in which two reads are mapped may also be referred to as a signature. Genomic structure variations are detected using the types and mapping forms of such signatures.

It may be more effective to detect a structural variation using a plurality of signatures than to compute a location where a structural variation has occurred using one signature. A clustering technique classifies (clusters) a plurality of signatures and computes a location of a structural variation that is representative of one cluster. The clustering technique can improve the reliability of prediction by removing a portion that is accidentally mapped. At this time, locations of both ends where a variation has occurred are called breakpoints. The clustering technique may be classified into several techniques depending on a signature determination method and an actual breakpoint computation method. For example, the clustering technique includes a standard clustering approach, a soft clustering approach, and a distribution-based clustering approach.

There are also analysis methods different from the PEM technique. For example, there is a technique for detecting a structural variation based on depth of coverage (DOC). However, the DOC-based analysis method has difficulty in detecting a signature in a small area and has limitations in determining breakpoints.

Meanwhile, there are commercial programs that detect genomic structural variations based on NGS. For example, the programs include MoDIL, SeqSeq, PEMer, VariationHunter, Pindel, BreakDancer, ABI SOLiD software Tool, etc. The tools differ in terms of detectable signatures, a clustering method for detecting signatures, or a method of constructing and processing a window.

For convenience of description, it is assumed that the NGS-based genome analysis technique uses PEM. However, the method of detecting structural variations, which will be described below, is not limited to a specific genome analysis methodology.

Sample data, sample sequence data, or sample genome data refer to genome data of a target to be analyzed. For example, the sample sequence data may be genome data of a patient with a specific disease. The sample data may be genome data of a cancer patient (suspect). The sample sequence data is a result of the NGS apparatus analyzing the sequence. Accordingly, the sample sequence data has an NGS analysis data format. For example, the sample sequence data may be a file in a format such as “fastq.”

Reference data, reference sequence data, or reference genome data refer to data to be compared for analysis of the sample sequence data. A structural variation in the sample sequence data may be detected by comparing the difference between the sample sequence data and the reference genome data. The reference genome data is data prepared in advance through experimental results. As will be described later, there are pieces of reference genome data for various races. Also, the pieces of reference genome data differ from each other in terms of completeness. Reference genome data completed by many research institutes over a long period of time has a high degree of completeness. Here, the completeness may be a ratio (proportion) of a sequenced portion to the whole genome sequence. When there are many sequenced parts, it can be said that the degree of completion is relatively high. There is a piece of reference genome data having a degree of completion greater than or equal to a specific reference value. For example, here, the reference value may be 90%.

Standard genome data has a similar meaning to the reference genome data. However, the standard genome data is basically defined as single reference genome data published through research. For example, genome data such as hg19 may be standard genome data.

Multi-reference genome data is a reference genome data set constructed with a plurality of pieces of reference genome data. The multi-reference genome data may be constructed using comparative data (dbSNP, etc.) and filtering out analysis errors and reference genomes of various races. The multi-reference genome data will be described below.

The following description assumes that genomic structural variations are analyzed through a computer apparatus. A computer apparatus refers to a device that can calculate and process certain data, such as a personal computer (PC), a smart device, and a network server. The computer apparatus that analyzes genomic structural variations may be referred to as a structural variation detection apparatus. The computer apparatus and the structural variation detection apparatus will be described below. For convenience of description, the following description assumes that a computer apparatus performs each process of the analysis of the genomic structural variations.

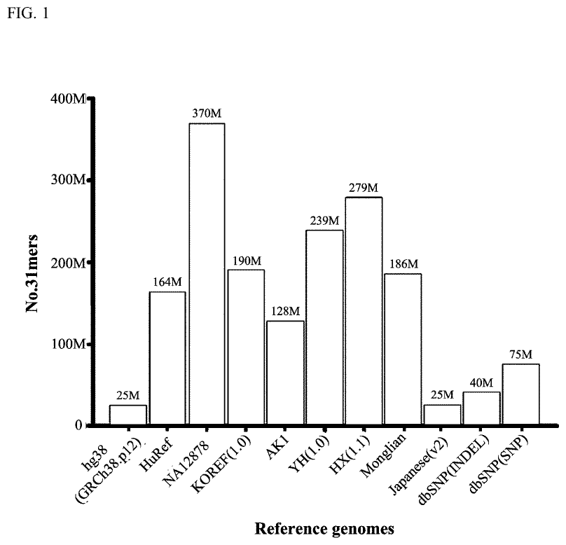

FIG. 1 is a result of comparing a 31-mer of the hg19 reference genome to 31-mers of reference genomes of various races. FIG. 1 is a result of comparing the 31-mers of the reference genomes of different races based on the hg19 reference genome. As the reference genomes of different races, hg38, HuRef, NA12878, KOREF, AK1, YH, HX, Mongolian, Japanese, dbSNP(INDEL), and dbSNP(SNP) were used. FIG. 1 is a result of calculating the number of specific 31-mers, which are not included in the hg19 reference genome, of different race reference genomes. Referring to FIG. 1, the numbers of 31-mers that are included in the reference genomes of the different races and that are not included in hg19, which is a representative reference genome of westerners, range from a minimum of 25 million to a maximum of 370 million. When the sequence differences between individuals and between races are not reflected, it is difficult to accurately perform genome analysis. A structural variation analysis method, which will be described below, uses multi-reference genome data to perform genome analysis without errors between individuals and between races.

First, the construction of the multi-reference genome data will be described. The multi-reference genome data should be prepared prior to the analysis of sample sequence data. The multi-reference genome data is prepared by the computer apparatus processing certain data.

(1) Basically, the multi-reference genome data includes reference genomes of a plurality of races. For example, the multi-reference genome data includes hg19, hg38, HuRef, NA12878, KOREF(1.0), AK1, YH(1.0), HX(1.1), Mongolian genome, Japanese genome(v2), and the like. The reference genome data of the plurality of races is intended to resolve interpretation errors occurring due to a sequence difference between races.

(2) Furthermore, the multi-reference genome data may further include dbSNP(INDEL), dbSNP(SNP), and reference genomes produced by users. dbSNP(INDEL) and dbSNP(SNP) are intended to resolve interpretation errors due to a sequence difference between individuals. The data may be referred to as data for filtering genomes.

The multi-reference genome data is constructed with a plurality of pieces of genome information, and a data structure for managing a plurality of pieces of genome data is necessary. To this end, the multi-reference genome data is composed of k-mers of the dbSNP data and the reference genomes of the plurality of races. Furthermore, the multi-reference genome data may be expressed as a hash table for a great deal of k-mers. For example, the multi-reference genome data may use, as a data structure, a hash table structure such as Sparsepp/KMC.

(3) Meanwhile, the multi-reference genome data may additionally use normal sequence data (NGS analysis result data of normal people). As the NGS analysis result, the normal sequence data may be data in a format such as fastq. When the hash table constructed with k-mers of the dbSNP data and the reference genomes of the plurality of races has normal sequence data, k-mers of the normal sequence data are included in the hash table. Here, k is a natural number of a certain size. For example, k may be 31.

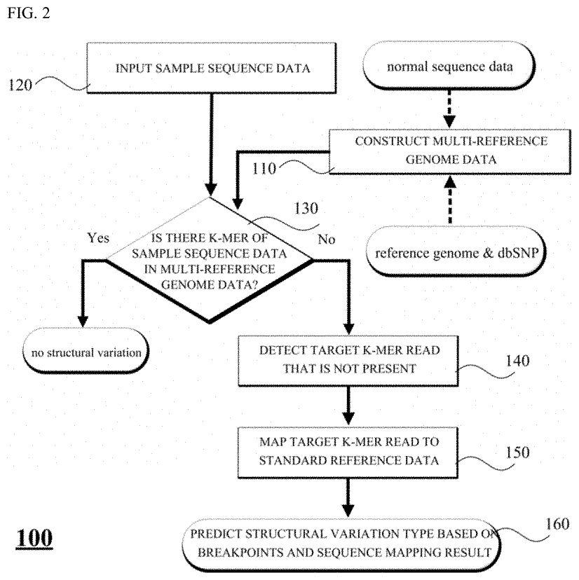

FIG. 2 is an exemplary flowchart of a process of detecting genomic structural variations based on a multi-reference genome. A computer apparatus constructs multi-reference genome data in advance (110). As described above, the computer apparatus constructs a k-mer data structure using the reference genomes of the plurality of races, published single nucleotide polymorphism (SNP) data, and published small insertions/deletions (INDEL) data. dbSNP(SNP) may be used as the published SNP data. dbSNP(INDEL) may be used as the published INDEL data. As described above, the computer apparatus pre-generates a k-mer hash database (multi-reference genome data) from a plurality of reference genomes, dbSNP information, and the like and loads the generated multi-reference genome data.

The computer apparatus receives sample sequence data to be analyzed (120). The sample sequence data is an NGS analysis result. The sample sequence data may be in a format such as fastq. The sample sequence data may be a genome analysis result for a patient or a suspected patient (hereinafter referred to as a user). The sample sequence data includes sequence analysis data derived from a user's diseased tissue (e.g., a tumor). Also, the sample sequence data may include sequence analysis data derived from a user's blood. The sample sequence data may include all the sequence analysis data derived from the user's tissue and blood.

By using a hash table of the constructed multi-reference genome data, the computer apparatus determines whether a sample sequence data read is present in the hash table (130). This process may be referred to as a process of filtering the sample sequence data using the multi-reference genome data. The computer apparatus may determine that a k-mer read in the hash table among reads of the sample sequence data is a part having no structural variation (yes in 130). On the contrary, the computer apparatus may analyze the type of structural variation on the basis of a k-mer read that is not present in the hash table among the reads of the sample sequence data (no in 130).

The computer apparatus detects the k-mer read that is not included in the hash table among the reads of the sample sequence data (140). Among the reads of sample sequence data, the k-mer read that is not included in the hash table is hereinafter referred to as a target k-mer read.

Then, the computer apparatus compares the target k-mer read to other reference genome data (150). The computer apparatus maps the target k-mer read to standard reference data (150). In this case, the standard reference data may use one piece of reference genome data with a high degree of completeness. For example, hg19 or hg38 may be used as the standard reference data. Alternatively, when the user is of a specific race, reference data of the corresponding race may be used. For example, in the case of structural variation analysis for Korean, KOREF may be used as the standard reference data. In some cases, furthermore, the standard reference may be composed of one or more pieces of reference data. It is assumed that hg19, which is a piece of reference genome data with a relatively high degree of completeness is used.

The computer apparatus maps the target k-mer read to hg19. The computer apparatus predicts a structural variation type for a sample on the basis of a result of the mapping to the standard reference data (e.g., hp19) (160). The computer apparatus may calculate a breakpoint list by mapping the target k-mer read and the standard reference data. Also, the computer apparatus may calculate a sequence matching result (signature) by mapping the target k-mer read and the standard reference data. Finally, the computer apparatus may predict a structural variation type for the sample sequence data on the basis of the breakpoint list and a feature, form, or pattern (signature) of the sequence matching. A criterion for predicting the structural variation type using breakpoints and the sequence mapping result may be similar to conventional structural variation detection techniques. All of the structural variation types may be predicted using the breakpoints and the sequence mapping result.

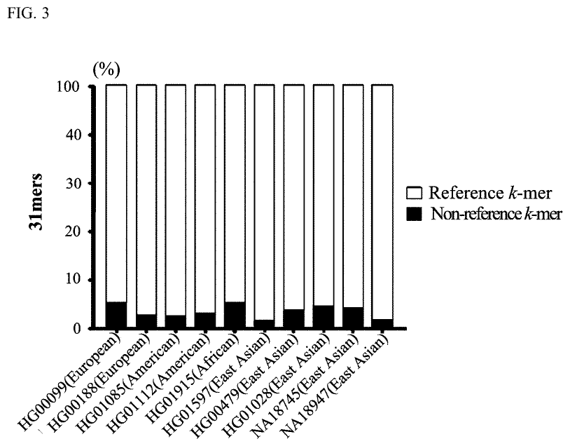

FIG. 3 is an example of a k-mer filtering result for samples of the 1000 Genomes Project. FIG. 3 is a result of filtering 10 k-mers in the 1000 Genomes Project. FIG. 3 shows that it is possible to effectively filter output information that causes errors in an analysis when multi-reference genome data is used. To this end, germline and somatic samples were used. In the bar plots of FIG. 3, “Reference k-mer” indicates a removed k-mer, and “Non-reference k-mer” indicates a k-mer remaining after filtering. A non-reference k-mer corresponds to the above-described k-mer read. Referring to FIG. 3, it can be seen that k-mers with information irrelevant to structural variations can be effectively removed from all the samples through k-mer filtering.

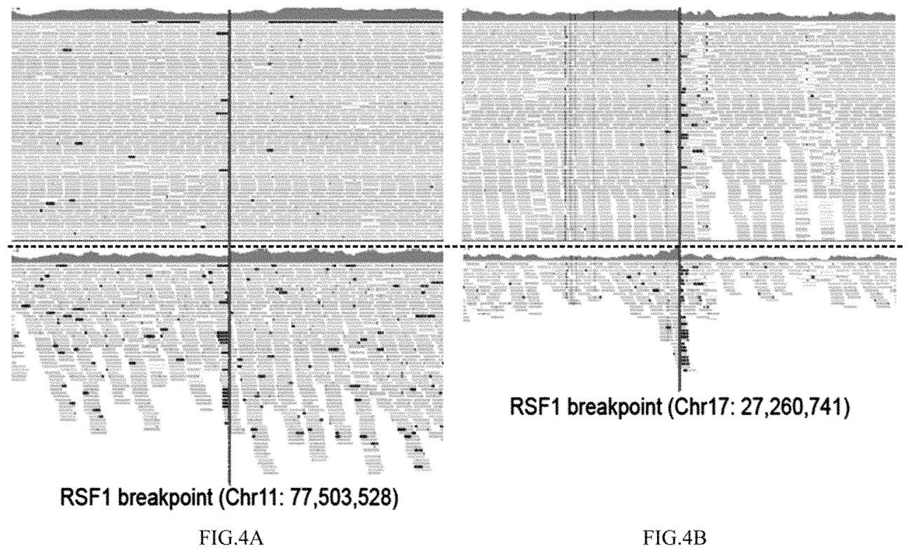

FIG. 4 is an example of a k-mer filtering result for a breast cancer sample verified to have structural variations. FIG. 4 is a filtering result for RSF1-PHF12 chromosomal rearrangement locations of a TCGA-A1-A0SM sample (breast cancer). FIG. 4 shows a result of mapping hg19 to the entire data and a result of mapping hg19 to data on which k-mer filtering has been performed. FIG. 4A is an example for Chromosome 11, and FIG. 4B is an example for Chromosome 17. In FIG. 4, a structural variation is a result of RSF1-PHF12 chromosomal rearrangement among 11 structural variations of the corresponding sample. In FIGS. 4A and 4B, regions above dotted lines indicate results before the k-mer filtering. The regions above the dotted lines indicate results of mapping the entire data to hg19. In FIGS. 4A and 4B, regions below the dotted lines indicate results after the k-mer filtering. The regions below the dotted lines are results of mapping only the target k-mer read to hg19 after the k-mer filtering.

In FIG. 4, a vertical solid line indicates breakpoints. Data providing breakpoint information related to structural variations is expressed in black. Referring to FIG. 4, it can be seen that data with erroneous information around breakpoints is effectively removed after the k-mer filtering. Also, the data providing breakpoint information related to structural variations can be more easily distinguished.

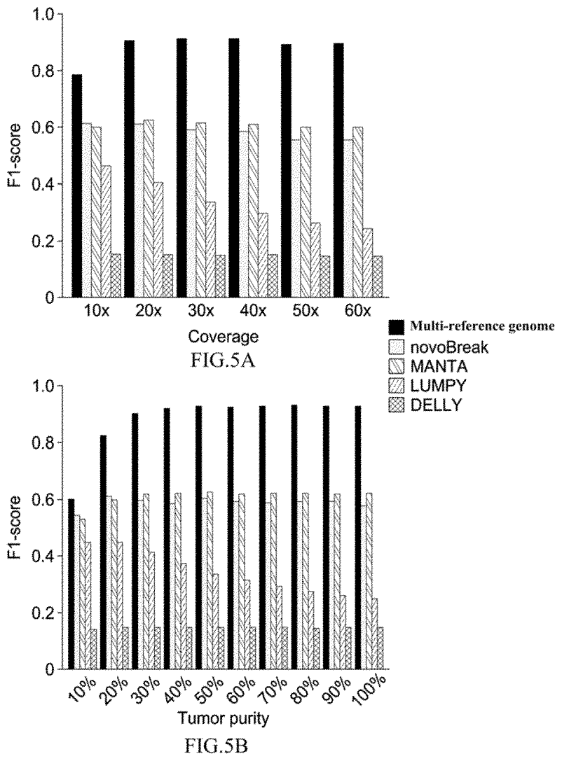

FIGS. 5 to 7 shows the effects of the above structural variation detection technique (according to the present disclosure) using the multi-reference genome data. The structural variation detection technique of the present disclosure is denoted as “multi-reference genome.” To verify the effects, a data set in which a structure variation is artificially generated was used. Also, this is an example of experimental results showing the accuracy of structural variation prediction according to sequencing depth or tumor purity. Sequencing depth and tumor purity have the greatest impact on performance when detecting structure variations. Generally, it is known that structural variation detection performance is lowest when the sequencing depth is 10× and the tumor purity is 10%.

The effects of this technique according to the present disclosure were compared to those of conventional predictive programs. The experiment results (data) for the structural variation detection technique of the present disclosure are indicated in black. The structural variation detection technique of the present disclosure uses a result of mapping to the standard reference genome after the k-mer filtering. The experiment results below are intended to determine whether an accurate structural variation type can be predicted through this process. It is verified whether it is possible to effectively detect various types of structural variations using the structural variation detection technique of the present disclosure. A total of 555 types of structural variations such as deletions, inversions, translocations, and duplications were used to conduct performance tests together with commercial programs.

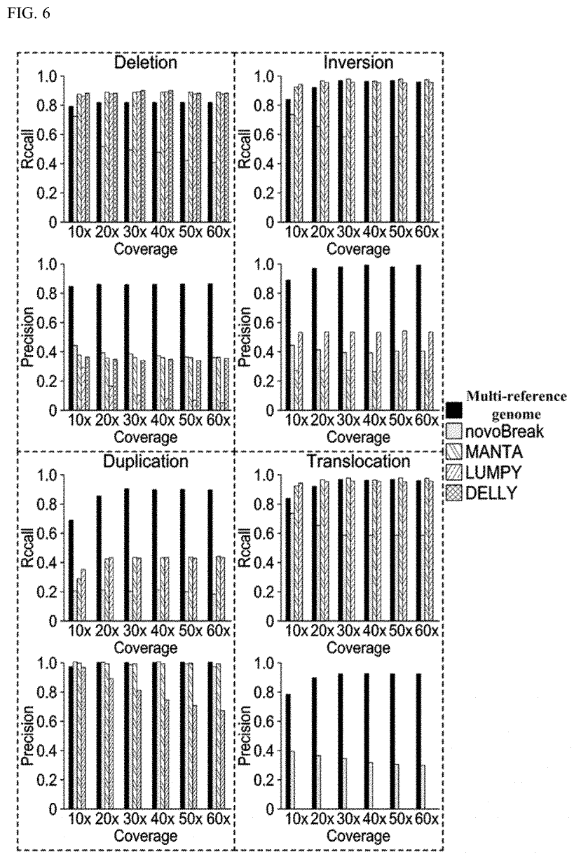

FIGS. 5A and 6 show the effects of the structural variation detection technique of the present disclosure according to various sequencing depths. For the experiment, a data set with sequencing depths of 10× to 60× was made. NOVOBREAK, LUMPY, SvABA, MANTA, and DELLY were used as the conventional predictive programs. As a result, FIG. 5A shows that when a structural variation is detected, the structural variation detection technique of the present disclosure exhibited an F1-score of 0.78, which is the best performance, even in the result of the sequencing depth of 10× in which the performance is lowest and exhibited an F1-score of 0.92 because the performance is improved along with the increase of the depth.

FIG. 6 shows the accuracy of prediction for various structural variations in the results of the sequencing depths. Referring to FIG. 6, the structural variation detection technique of the present disclosure shows the best performance for all types of structural variations.

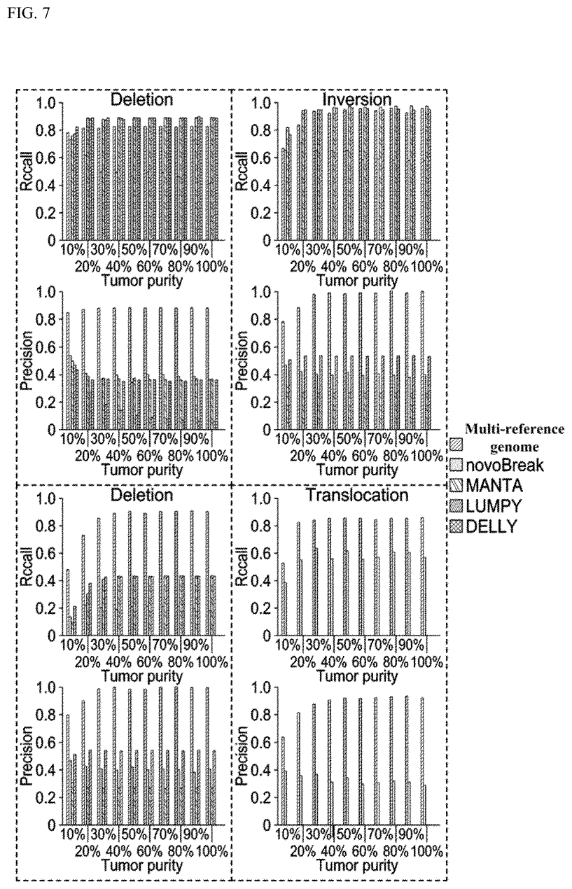

FIGS. 5B and 7 show the effects of the structural variation detection technique of the present disclosure according to various tumor purities. For the experiment, a data set having tumor purities from 10% to 100% was made by mixing normal genome information and genome information reflecting structural variations. Referring to FIG. 5B, the structural variation detection technique of the present disclosure exhibited an F1-score of 0.59 even at a tumor purity of 10% at which detection is most difficult (a condition in which information reflecting structural variations in a cancer genome is weakest). The structural variation detection technique of the present disclosure exhibited much better performance in consideration of the fact that NOVOBREAK, which exhibited the best performance among the conventional techniques, exhibited an F1-score of 0.48 (MANTA: 0.34, LUMPY: 0.38, and DELLY: 0.14).

FIG. 7 shows the accuracy of prediction for each structural variation in the results of the tumor purities. FIG. 7 shows that the structural variation detection technique of the present disclosure exhibits the highest precision and recall for most structural variation types even at a purity of 10% in the same manner as the depth-specific results.

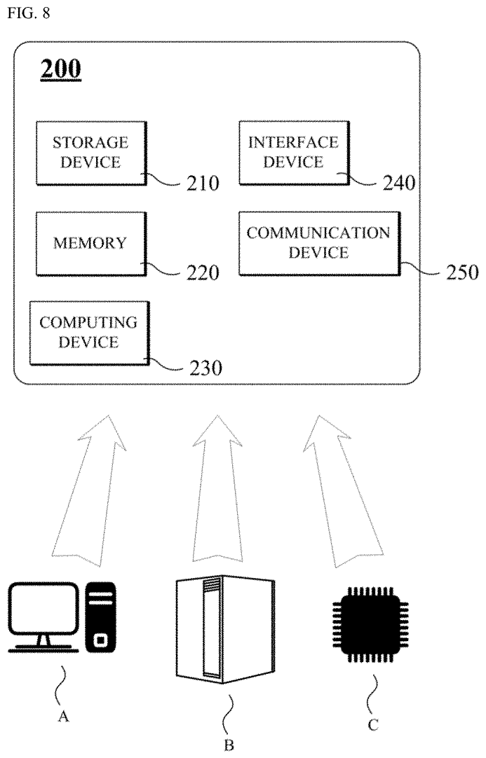

FIG. 8 is an example of a structure of a structural variation detection apparatus 200. FIG. 8 shows an apparatus for detecting a structural variation using the above-described multi-reference genome data. FIG. 8 corresponds to the above-described computer apparatus. The structural variation detection apparatus may be physically implemented in various forms. For example, as shown in the lower portion of FIG. 8, the structural variation detection apparatus may be implemented in a form such as a PC (A), a network server (B), and a dedicated analysis chipset (C).

The structural variation detection apparatus 200 includes a storage device 210, a memory 220, a computing device 230, an interface device 240, and a communication device 250.

The communication device 250 refers to a component for receiving and transmitting certain information through a wired or wireless network. The communication device 250 may receive sample sequence data, multi-reference genome data, or data for constructing multi-reference genome data (a plurality of pieces of reference genome data, dbSNP data, etc.) from an external object. The communication device 250 may receive certain data from a user terminal, an NGS analysis device, an NGS analysis server, etc. The communication device 250 may transmit structural variation type analysis results to a user terminal, a separate server, or the like.

The storage device 210 may store a program (code) for implementing the above-described structural variation analysis technique. The storage device 210 may store the multi-reference genome data, the sample sequencing data, etc. The memory 220 may store information received by the node apparatus 200 or data temporarily generated according to the operation of the computing device 230.

The interface device 240 is a device for receiving a certain instruction from an external user. The interface device 240 may receive a program or data basically required for operation of the node apparatus 200 from an input device or an external storage device that is physically connected to the interface device 240. For example, the interface device 240 may receive sample sequence data to be analyzed. Also, the interface device 240 may receive the multi-reference genome data. Also, the interface device 240 may receive various pieces of reference data to construct the multi-reference genome data.

The communication device 250 and the interface device 240 are devices that receive certain data or instructions from the outside. The communication device 250 and the interface device 240 may be referred to as input devices.

The computing device 230 may generate multi-reference genome data using data input from an input device or data stored in the storage device 210. The computing device 230 may compare the multi-reference genome data and the sample sequence data and determine at least one target k-mer read that is not included in the multi-reference genome data among reads of the sample sequence data. The computing device 230 may predict a structural variation type on the basis of breakpoints and a candidate region of the structural variation determined by mapping the at least one target k-mer read to the standard reference genome data. The computing device 230 may be a device for processing data and performing certain computations, such as a processor, an application processor (AP), and a chip with an embedded program.

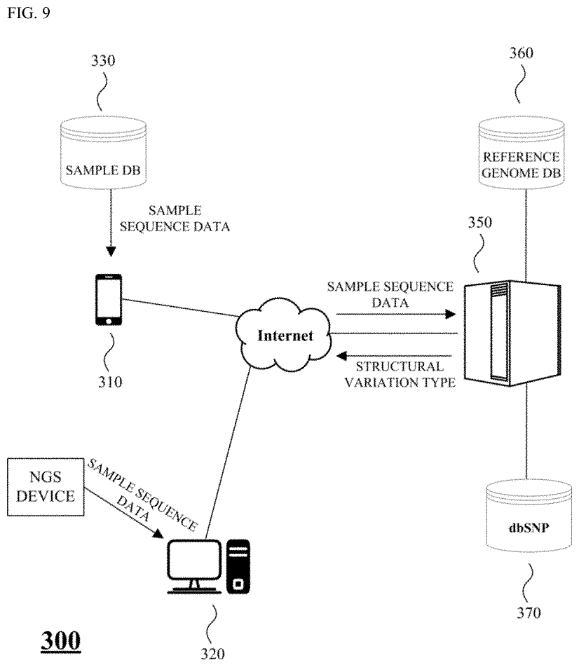

FIG. 9 is an example of a structural variation detection system 300. FIG. 9 shows an embodiment in which a genomic structural variation analysis service is provided using a network. The system 300 includes user terminals 310 and 320 and a service server 350. The user terminals 310 and 320 correspond to client devices. In FIG. 9, the service server 350 corresponds to the above-described structural variation detection apparatus. In FIG. 9, detailed descriptions of security or communication between objects are omitted. Each object may perform certain authentication before performing communication. For example, only a user who has been successfully authenticated can request the service server 350 to analyze structural variations.

The user may request the service server 350 to analyze genomic structural variations through a user terminal. The user may receive sample sequence data from a sample DB 330. The sample DB 330 stores an NGS analysis result for a specific user. The sample DB 330 may be an object located in a network. Alternatively, the sample DB 330 may be a simple storage medium. The user delivers the sample sequence data to the service server 350 through the user terminal 310. When receiving the analysis request including the sample sequence data, the service server 350 predicts a structural variation type for the sample sequence data through the above-described process. It is assumed that the service server 350 constructs the multi-reference genome data for analysis and acquires the standard reference genome data in advance. The service server 350 may receive the reference genome data from a reference genome DB 360. The service server 350 may receive SNP and INDEL data from dbSNP 370. The service server 350 may construct the multi-reference genome data using the dbSNP and a plurality of pieces of reference genome data by the above-described method. The service server 350 may transmit a generated structural variation analysis result to the user terminal 310. Alternatively, although not shown, the service server 350 may store the structural variation analysis result in a separate storage medium or may deliver the structural variation analysis result to a separate object.

In the NGS analysis process, the user may deliver the sample sequence data to the service server 350 through the user terminal 320. The user terminal 320 may receive the sample sequence data from the NGS analysis apparatus. When receiving the analysis request including the sample sequence data, the service server 350 predicts a structural variation type for the sample sequence data through the above-described process. It is assumed that the service server 350 constructs the multi-reference genome data for analysis and acquires the standard reference genome data in advance. The service server 350 may transmit a generated structural variation analysis result to the user terminal 320. Alternatively, although not shown, the service server 350 may store the structural variation analysis result in a separate storage medium or may deliver the structural variation analysis result to a separate object.

Also, the above-described genomic structural variation detection method may be implemented using a program (or application) including an executable algorithm that may be executed by a computer. The program may be stored and provided in a non-transitory computer-readable medium.

The non-transitory computer-readable medium refers a medium that semi-permanently stores data and is readable by a device rather than a medium that temporarily stores data such as a register, a cache, and a memory. Specifically, the above-described various applications or programs may be provided while being stored in a non-transitory computer-readable medium such as a compact disc (CD), a digital versatile disc (DVD), a hard disk, a Blu-ray disc, a Universal Serial Bus (USB), a memory card, a read-only memory (ROM), etc.

The above embodiments and drawings attached to the present specification are merely intended to clearly describe part of the technical spirit included in the present invention, and it is apparent that all modifications and detailed embodiments that can be easily derived by those skilled in the art within the scope of the technical spirit included in the specification and the drawings of the present invention are included in the scope of the invention.

1. A method of detecting a genomic structural variation based on k-mer set, the method comprising:
receiving, by a computer apparatus, sample sequence data;
filtering out, by the computer apparatus, k-mer set in reference genome data from the sample sequence data to extract at least one target k-mer read from reads of the sample sequence data;
determining, by the computer apparatus, a breakpoint and a candidate region of a structural variation by mapping the at least one target k-mer read to standard reference genome data; and
predicting, by the computer apparatus, a structural variation type for the sample sequence data on the basis of a sequence mapping pattern and the breakpoint in the mapping result,
wherein the reference genome data comprise reference genomes of a plurality of races.

2. The method of claim 1, wherein the k-mer set includes all k-mers from the reference genome data.

3. The method of claim 1, wherein the reference genome data further includes single nucleotide polymorphism (SNP) data and small insertions/deletions (INDEL) data.

4. (canceled)

5. The method of claim 1, wherein the reference genome data further includes at least one k-mer of normal genome sequence of a normal person.

6. The method of claim 1, wherein data structure of the k-mer set is a hash table.

7. The method of claim 1, wherein the sample sequence data is genome sequence data of a patient.

8. The method of claim 1, wherein the standard reference genome data is reference genome data with a degree of genome sequence completeness greater than or equal to a reference value.

9. The method of claim 1, wherein the standard reference genome data is at least one of hg19, hg38, and KOREF.

10. A computer-readable recording medium having a computer program recorded thereon to execute the method of any one of claims 1 to 3 and 5 to 9.

11. An apparatus for detecting a genomic structural variation based on a multi-reference genome, the apparatus comprising:
an input device configured to receive sample sequence data;
a storage device configured to store reference genome data and standard reference genome data; and
a computing device configured to
filter out k-mer set in the reference genome data from the sample sequence data to extract at least one target k-mer read from reads of the sample sequence data
predict the structural variation type on the basis of a sequence mapping pattern and a breakpoint determined by mapping the at least one target k-mer read to the standard reference genome data,
wherein the reference genome data comprise reference genomes of a plurality of races.

12. The apparatus of claim 11, wherein the reference genome data further includes single nucleotide polymorphism (SNP) data and small insertions/deletions (INDEL) data.

13. The apparatus of claim 11, wherein the reference genome data further includes normal genome sequence of a normal person.

14. The apparatus of claim 11, wherein the standard reference genome data is reference genome data with a degree of genome sequence completeness greater than or equal to a reference value.

15. The apparatus of claim 11, wherein the standard reference genome data is at least one of hg19, hg38, and KOREF.
